(19) United States
(12) Patent Application Publication
(10) Pub. No.: US 2014/0275865 A1
(43) Pub. Date: Sep. 18, 2014

(54) SHEATHED RIVET

(71) Applicant: Microtech Medical Technologies Ltd., Tel Aviv (IL)

(72) Inventors: Eric S. TAMMAM, Modiin (IL); Oleg WEIZMAN, Herzliya (IL); Yekaterina DLUGACH, Beer-Sheba (IL); Nadav AGIAN, Kfar Yona (IL)

(21) Appl. No.: 13/935,001

(22) Filed: Jul. 3, 2013

Related U.S. Application Data
(60) Provisional application No. 61/793,549, filed on Mar. 15, 2013.

(51) Int. Cl. A61B 5/00; A61B 5/145; B21D 31/00; A61B 5/0215

(52) U.S. Cl. CPC A61B 5/6882; A61B 5/0215; A61B 5/14503; B21D 31/00
USPC 600/309; 606/151; 600/486; 264/266; 72/342.1

(57) ABSTRACT

The device and method of the invention generally relate to a system and method for percutaneous delivery, implantation and securement of an anchor at a target site. The system comprises an anchor having a bridge, a first stabilizer having a crimped state and a deployed state, a second stabilizer having a crimped state and a deployed state, and a positioning arm. The system may further comprise a cannula, pushrod, and sheath. The system permits the deposit of an anchor at a target location in the body by utilizing a controlled amount of force. The anchors and methods are particularly well-suited to implantation within the body of a living animal or human to monitor various physiological conditions.

### FIELD OF INVENTION

The present invention relates to a system and method for implantation and securement of a small implantable element to monitor and/or treat physiological conditions of the body. In addition, the invention describes a novel anchor to position a small implantable element in a desired position within a wall of a target tissue of the body. The invention also relates to a method for implanting the anchor directly in a target wall of the body and securing the anchor in a fixed location without displacement over the life of the anchor.

### BACKGROUND

Percutaneously-delivered deployment systems are used to embed implantable devices within a lumen of the body. Generally, such a deployment system comprises a catheter, an implantable device, and an element for releasing the implantable device at the target location, for example, described in U.S. Pub. No. 2003/0125790 and U.S. Pub. No. 2008/0071248. The catheter houses the deployment system and permits the system to be advanced to the target location, where the implantable device is released. The implantable device remains within the body to perform its intended function after the deployment system is retracted.

Importantly, the implantable device must be securely attached to the target location before the deployment system releases the device. A device which is not securely embedded may become dislodged and pose serious risks to the patient, especially if the device migrates from the implantation site. An insufficiently secured device that circulates in the body may cause serious injuries, including an acute myocardial infarction, a stroke, or organ failures. Thus, there is a need for an anchor and a deployment system that assures that the device is implanted and secured in the body in a fixed location without dislocation over the life of the device. Also, there is a need for an anchor that permits the deployment of the implantable device with minimal damage to the wall of the organ of the body. Further, there is a need for an anchor that permits the desired orientation of the device relative to the lumen of the body without relocating or adjusting the implanted device once deployed within the lumen.

Such an anchoring system is advantageous to the clinician in that it enables the implantation and securement of an implantable device at the desired orientation through a cannula-based delivery system for ease of deployment while reducing the risk of displacement of the device over time. Further, such a system can eliminate the need for a follow-up procedure to retrieve the dislodged implantable device, as is the case where the device is not securely implanted through a reliable means, or to relocate the implantable device in order to establish the desired orientation of the device relative to the lumen. For example, current procedures for monitoring hepatic portal pressure and the detection of malignant hypertension are not satisfactory and generally involve an indirect measurement of the portal venous pressure through the hepatic venous system due to the difficulty in accessing the target site for the direct implantation of the monitoring device. Moreover, there are no current procedures for implanting a monitor. Thus, a system that is capable of reliably and securely implanting a detector of portal pressure, for example, could reduce the complexities of the procedure and the need for post-operative treatments, providing favorable outcomes for the patient.

A need therefore exists for an anchoring system that allows for simple, safe and secure implantation of a device in a fixed location, while orienting the device as desired.

### SUMMARY OF THE INVENTION

The present invention relates to an anchor and a deployment system for the percutaneous delivery and secure implantation of a device in the wall of a target tissue of the body to measure or treat various bodily conditions. The anchor comprises a first stabilizer, a second stabilizer, a first ring, a second ring, a bridge there between, and a positioning arm. The first and second rings are attached to each end of the bridge. The first stabilizer extends from the first ring and the second stabilizer extends from the second ring. One of said stabilizers is located at the proximal end of the anchor and may be referred to as a proximal stabilizer, and the other stabilizer is located at the distal end of the anchor and may be referred to as a distal stabilizer. The first and second stabilizers may transition from a crimped position to a deployed position upon deployment in a target site wall. The stabilizers are characterized by their distinct crimped and deployed configurations. The crimped configuration is characterized in that the stabilizers fit inside the delivery system. The deployed configuration is characterized as having stabilizers that extend in a direction substantially perpendicular to the bridge of the anchor to a diameter sufficient to hold the anchor in position in the wall; that is, the stabilizers extend in a direction generally parallel to the target tissue wall. The positioning arm may house a sensor or other small implantable element which may be strategically positioned, for example, to protrude into the target site after deployment.

The invention also relates to a system for deploying the anchor comprising an introducer cannula, a pushrod, a sheath and an anchor. The deployment system may deliver the anchor directly to the target site wall (i.e. extra-luminally) or use a catheter-based system (i.e. intra-luminally).

Further, the invention relates to a method of deploying the anchor into a vessel wall comprising introducing the cannula into the target site wall so that the tip of the cannula is in the inner portion of the target site wall; positioning the crimped anchor with a pushrod between the inner and outer portion of the target site wall; releasing the first stabilizer of the anchor so that the first stabilizer expands from a partially deployed anchor; releasing the second stabilizer of the anchor so that the second stabilizer expands forming the fully deployed anchor; and retracting the cannula. Further, the invention relates to a method of manufacturing the anchor by, for example, use of a mandrel specifically designed for the manufacturing of an anchor according to the principles of this invention.

In another aspect of the invention, the invention includes a mandrel for manufacturing the implantable anchor having first and second stabilizers. The mandrel comprises a first disc, a second disc and an axel therebetween. Each disc may have a groove extending in a shape from the axel in which a material forming the anchor is placed. Mandrel coverings may also be used to encase the mandrel during the treatment processes.

The present invention provides the advantages of a shortened procedure time, lessened procedural discomfort, increased procedural success, and increased safety. The invention presents the further advantage of enabling implantation of a detector without necessitating x-ray or ultrasound imaging for guidance.

### BRIEF DESCRIPTION OF THE DRAWINGS

FIG. 1A shows an anchor in accordance with this invention in a fully crimped state.

FIG. 1B shows the anchor of FIG. 1A in a fully deployed state.

FIG. 2A shows an anchor in accordance with this invention in a fully crimped state.

FIG. 2B shows the anchor of FIG. 2A in a fully deployed state.

FIG. 3A shows an anchor in accordance with this invention in a fully crimped state.

FIG. 3B shows the anchor of FIG. 3A in a fully deployed state.

FIG. 4 shows an anchor in accordance with this invention in a crimped state.

FIG. 5 shows an anchor in accordance with this invention in a crimped state.

FIG. 6 shows an anchor in accordance with this invention in a crimped state.

FIG. 7 shows a ring and stabilizers in accordance with this invention in a crimped state.

FIG. 7A shows various perspectives of the ring and stabilizers of the embodiment of FIG. 8 in a deployed state.

FIG. 8 shows various embodiments of rings and stabilizers arranged on a ring in the deployed state.

FIG. 8A shows a top view of various stabilizers and ring embodiments in accordance with this invention.

FIG. 9A shows one embodiment of an anchor rod, second ring and first ring in accordance with this invention.

FIG. 9B shows the anchor rod, second ring and first ring of FIG. 10A in an extended state.

FIG. 9C shows another embodiment of the anchor rod, second ring and first ring in accordance with this invention.

FIG. 9D shows the anchor deployed at a target location having a thin tissue wall.

FIG. 9E shows the anchor deployed at another target location having a thick tissue wall.

FIG. 10 shows an introducer cannula loaded with the anchor in accordance with the invention, introduced into a wall at the target site.

FIG. 10A shows the introducer cannula of FIG. 12 wherein the anchor is in a state of partial deployment, with the first stabilizer and positioning arm deployed in the inner portion of the target site wall.

FIG. 10B shows the introducer cannula of FIG. 13 wherein the anchor is in a state of full deployment, with the second stabilizer deployed outside the vessel wall and the first stabilizer and positioning arm deployed in the inner portion of the target site wall.

FIG. 11 shows an introducer cannula loaded with the anchor in accordance with this invention.

FIG. 12A shows a release mechanism in accordance with this invention in a pre-deployment state.

FIG. 12B shows the release mechanism of FIG. 12A in a state of partial deployment with the sheath refracted from the pushrod.

FIG. 13A shows a release mechanism in accordance with this invention.

FIG. 13B shows the release mechanism of FIG. 13A in a state of partial deployment with the aperture of the pushrod rotated to align with a member of a stabilizer.

FIG. 14 shows a flat metal pattern of the anchor of FIG. 1A prior to formation into an anchor in accordance with this invention.

FIG. 15 shows a flat metal pattern of the anchor of FIG. 3A prior to formation into an anchor in accordance with this invention.

FIG. 16 shows a flat metal pattern prior to formation into an anchor in accordance with the invention.

FIG. 17 shows one embodiment of an anchor in a deployed state in accordance with this invention.

FIG. 18A shows one embodiment of a mandrel designed to manufacture an anchor in accordance with the invention.

FIG. 18B is the front view of the mandrel of FIG. 18A.

FIGS. 18C and 18D show the mandrel covering(s) for use with the mandrel of FIG. 18A.

The invention is discussed and explained below with reference to the accompanying drawings. The figures are provided as an exemplary understanding of the invention and to schematically illustrate particular embodiments and details of the invention. The figures are not necessarily drawn to scale. The skilled artisan will readily recognize other similar examples equally within the scope of the invention. The drawings are not intended to limit the scope of the invention as defined in the appended claims.

### DETAILED DESCRIPTION OF THE INVENTION

The invention generally relates to an anchor system, deployment system and method for percutaneously implanting an anchor in the body carrying a small implantable element. The system and method relate to particularly small anchors, e.g., between 0.005 to 100 mm³ in volume, which are implanted in the wall of target tissue in the body. The size parameters of the anchor will be defined by the thickness of the target tissue wall. Nonetheless, the anchor may have an outer diameter in the range of 0.01 to 10 mm, a height that is preferably no more than 20 mm, and may preferably be adapted to allow for the integration of a small implantable element having a diameter in the range of 0.01 to 10 mm and a height in the range of 0.01 to 20 mm. It may be desirable that the element is fully integrated into the positioning arm of the anchor. The anchor is composed of a non-thrombogenic, non-biodegradable and non-biofouling material, preferably, a shape-memory material such as, for example, Nitinol, stainless steel or other suitable alloys or polymers. In one embodiment, the anchor has an outer diameter of 1 mm, a height of less than 0.4 mm and allows for the integration of a small implantable element having a diameter of 0.8 mm and a height of 0.3 mm. One preferred target area for embedding the anchor, which may be based on the thickness of the blood vessels at the target site, may be no less than 0.5 mm and no greater than 50 mm. Target areas of non-vessel target structures include, for example, the septum in the heart or the parenchyma of the liver, which may have thicknesses in the range of 0.5 to 10 mm.

The size and relatively low invasiveness of such anchors make them particularly well suited to medical and physiological uses, including, but not limited to, measuring bodily fluids, such as for example in the blood vessel/artery/vein. Generally, such anchors may aid in measuring chemical or physical parameters of bodily fluids, such as occurs in the blood, urine or digestive fluids. Implants in the heart for example may be used for measuring left atrial pressure in congestive heart failure applications. Implants in the liver may be used for intra-abdominal pressure. Such anchors are also applicable, for example, to aid in monitoring particular diseases or conditions or body chemical or physiological parameters, to deliver a therapeutic agent or other similar situations.

The anchor comprises a first stabilizer, a second stabilizer, a bridge there between, a first ring, a second ring, and a positioning arm. Each of the first and second stabilizers comprise one or members that may be formed in various shapes, such as, for example, prongs, coils, helixes, etc. One such stabilizer may be located at the proximal end of the anchor (the proximal stabilizer) and the other such stabilizer may be located at the distal end of the anchor (the distal stabilizer). Each member of a first or second stabilizer has a crimped state, that may be orderly, such as for example in a coil or a straight prong in a crimped position, or alternatively may be an irregular shape having bends, loops or twists for example, or generally amorphous. Each member of a first or second stabilizer also has a deployed state, for example, a diameter greater than the diameter of the opening in the target site wall created by a cannula upon delivery of the anchor. The target site wall is understood as the tissue at the site of deployment through which the anchor protrudes for deployment. Preferably, the target site tissue is an organ having bodily fluid transported through it, for example, blood vessels, heart chambers, digestive organs, urinary tract organs, the liver and the like. The members of the first stabilizer extend from a first ring, and the members of the second stabilizer extend from a second ring. The first ring and second ring are connected by the bridge. The positioning arm may extend from either the first ring or the second ring. The ring may be of any shape, including, circular, and be either hollowed or filled.

The invention also relates to a deployment system for delivering an anchor, comprising an introducer cannula having an inner lumen, which houses a pushrod, a sheath and the anchor. In an alternative embodiment, the deployment system may include a catheter system for intra-luminal delivery of the anchor. In one embodiment, a sheath surrounds the crimped anchor and maintains the first and second stabilizers and positioning arm in a crimped position in the introducer cannula. Here, the first stabilizer and positioning arm may be oriented at the distal end of the anchor, and the second stabilizer may be oriented at the proximal end of the anchor once assembled onto the deployment system. The sheathed anchor is placed in the desired position using the cannula and pushrod such that the bridge traverses the target site wall with the first stabilizer and positioning arm protruding into the target site. Once in position, the sheath is retracted, releasing the first stabilizer and the positioning arm from their crimped position into the target site so that the positioning arm is positioned on one side of the target site wall. Next, the second stabilizer is released from its crimped position onto the other side of the target site wall, thereby securing the anchor in a fixed position traversing the target site wall.

In an alternative embodiment the positioning arm may be located at the proximal end of the anchor, thereby co-located with the second stabilizer. Here, the deployment system further comprises a catheter and the anchor is deployed intra-lumenally such that the anchor is positioned in a vessel wall target site from within a vessel, for example. In this embodiment, the cannula and pushrod position the distal end of the anchor at the target site wall so that the first stabilizer is on the outer side of the vessel wall. Next the sheath is pulled back allowing the first stabilizer to deploy, then the second stabilizer thereby deploying the anchor.

In yet another embodiment, the deployment system may comprise a catheter with the anchor disposed therein. The positioning arm may be located at the distal end of the anchor, co-located with the first stabilizer. In this embodiment, the catheter may be advanced intra-lumenally in a first vessel in order to access a target site located in a second, nearby vessel. Once the target site is reached, an access device, e.g., a needle, may be used to access the target site in the second vessel from the first vessel. Thereafter, the anchor may be deployed in the second vessel with the positioning arm extended within the second vessel. For example, the portal vein may be accessed from the hepatic vein (intrahepatic) by this method.

The present invention also comprises a method for using a deployment system comprising a cannula, sheath and anchor. The target location may be identified by fluoroscopy or ultrasound and accessed by well-known access routes. The method comprises the steps of (i) advancing the cannula to the target site wall; (ii) introducing the cannula into the target site wall so that the tip of the cannula is in the inner portion of the target site wall; (iii) advancing the crimped anchor to said target site through said cannula, thereby positioning the crimped anchor between the inner and outer portion of the target site wall; (v) administering a controlled amount of force to retract the sheath to deploy the anchor system; (vi) releasing the first stabilizer of the anchor so that the one or more members of the first stabilizer expand; (vii) releasing the second stabilizer of the anchor so that the one or more members of the second stabilizer expand; and (viii) retracting said cannula. An additional optional step comprises slightly retracting the delivery system to ensure the first stabilizer is flush against the vessel wall and deployed.

The invention also relates to an optional feature of the deployment system comprising an introducer cannula having an interior lumen that houses a pushrod and a sheath that covers the anchor, wherein the pushrod may have one or more apertures through which a member of a first or second stabilizer protrudes into the space between the pushrod and the cannula. The frictional force between the outer wall of the pushrod and the inner wall of the sheath holds the member in place until by one or more methods, the frictional force on the member is released for deployment. Thus, the method of deployment in connection with this embodiment further comprises the steps of (a) withdrawing the sheath holding the one or more members of the second stabilizer in place and (b) withdrawing the pushrod to release the one or more members of the second stabilizer through the one or more apertures of the pushrod. In an alternative embodiment, each of the one or more apertures of the pushrod consist of an “L” shape formed by an aperture neck and an aperture arm. In the pre-deployment position, a member of the second stabilizer may extend through the aperture arm. Thus, the method of deployment in connection with this embodiment further comprises the step of (a) rotating the pushrod such that the aperture neck aligns with the member of the second stabilizer, and (b) withdrawing the sheath to release the member of the second stabilizer.

The invention further comprises a method of manufacturing the anchor comprising the steps of (i) producing a wire from a suitable material; and (ii) applying a heat treatment, as needed, to the wire to conform the wire to a predetermined shape with the use of a mandrel. In one embodiment, the mandrel comprises a first disc having a groove, a second disc having a groove and an axel therebetween. Another method of manufacturing according to this invention comprises the steps of (i) laser cutting a preselected pattern from a flat metal sheet, such as, for example, Nitinol; and (ii) forming the pattern into an anchor having a first stabilizer, a second stabilizer and a bridge positioned therebetween through application of heat treatment, welding or mechanical force.

The following sections describe various exemplary illustrations of the invention. It is understood that these figures represent examples of the features of the invention but are not limiting. The skilled person will readily recognize other embodiments within the scope of the invention.

FIG. 1A illustrates one embodiment of the anchor of the invention. Anchor 110 comprises bridge 115, a first ring 119 and a second ring 118. Extending from the first ring 119 is a first stabilizer 125, which is coiled when in a crimped state, as well as a positioning arm 130 positioned within the coil formed by the first stabilizer 125. Extending from the second ring 118 is a second stabilizer 120 that is coiled when in a crimped state. The positioning arm 130 may extend from the first or second ring depending upon how the anchor is delivered. If delivered directly into the target site using a cannula only (e.g. extra-luminally), then the positioning arm is preferably attached to the first ring; whereas, if the anchor is delivered using a catheter-based system (e.g. intra-luminally), then the positioning arm is preferably attached to the second ring. The first stabilizer 125 and the second stabilizer 120 are each formed of a member in FIG. 1A. The member may be in the form of a wire, band, strip or other appropriate configuration so as to function as described herein. In the embodiment illustrated in FIG. 1, the member is formed by a strip. In FIG. 1A, the anchor 110 is in a pre-deployment state as in a delivery system, wherein the first stabilizer 125, second stabilizer 120 and positioning arm 130 are in a crimped state. In one embodiment, the ring associated with the positioning arm may be larger than the other ring or the opposite depending on the configuration of the pushrod.

The bridge 115 may be of any length necessary depending on the thickness of the target site wall. The length of the bridge will be determined to ensure that the first ring 119 and first stabilizer 125 can extend into the target site lumen, thus allowing the first stabilizer 125 to extend (in this embodiment, by partially unspooling from a crimped coil), while at the same time the second ring 118 and second stabilizer 120 remain outside the target site wall and the second stabilizer 120 is allowed to unspool as well. FIG. 1B illustrates anchor 110 of FIG. 1A in a fully deployed state. FIG. 1C illustrates anchor 110 implanted in vessel tissue 150. Bridge 115 extends

Alternatively, the first and second stabilizers 125, 120 may be designed to adapt upon deployment to variability in the thickness of the wall by—for example—extending at an angle relative to parallel to the target site wall on either side, such that the far end of each stabilizer is closer to the target site wall than the point at which each said stabilizer is attached to the ring, to compensate in the event that the bridge substantially exceeds the width of the target site wall.

FIG. 2A illustrates another embodiment of anchor 210 in a crimped state wherein the first stabilizer has a first member 225a and a second member 225b, as well as a second stabilizer having a first member 220a and a member 220b. The first member 225a and second member 225b of the first stabilizer are positioned around the first ring 219 and are coiled in parallel with each other. Likewise, the first member 220a and the second member 220b of the second stabilizer are positioned 180° apart on the second ring 218 and are coiled in parallel with each other. Alternatively, stabilizers of this embodiment may form the helical configuration illustrated in FIG. 2A wherein each of the first and second members of each stabilizer are formed from a continuous loop so that the helical coil of each stabilizer does not include blunt ends, i.e., each stabilizer forms a double helix. FIG. 2B illustrates anchor 210 in a fully deployed state, with each of the first and second members 225a, 225b of the first stabilizer and the first and second members 220a, 220b of the second stabilizer fully deployed. The deployed configuration is characterized as having stabilizers that extend in a direction substantially perpendicular to the bridge of the anchor so that the anchor is fully engaged, for example, to a diameter greater than the diameter of the opening in the target site wall created by the cannula upon delivery of the anchor.

FIG. 3A illustrates yet another embodiment of anchor 310 in a pre-deployment state, wherein the first stabilizer includes a first member 325a, a second member 325b and a third member 325c, and the second stabilizer includes a first member 320a, a second member 320b and a third member 320c. In the pre-deployed state, the first, second and third members 325a, 325b, 325c of the first stabilizer extend approximately straight from the first ring 318 in the direction of the second ring 319; and the first, second and third members 320a, 320b, 320c of the second stabilizer extend approximately straight from the second ring 318 in the direction of the first ring 319. In FIG. 3A, the members are shown extending from one ring approximately straight in the direction of the other ring. FIG. 3B illustrates anchor 310 in a fully deployed state, whereby the first, second and third members 325a, 325b, 325c of the first stabilizer and the first, second and third members 320a, 320b, 320c of the second stabilizer bend away from the bridge 315.

Other forms of first and second stabilizers may be utilized in connection with the anchor, as illustrated, for example, in FIG. 4, showing a first stabilizer 425 and second stabilizer 420 in a crimped state wherein the first and second stabilizers 425, 420 coil inward toward the center of the anchor 410. FIG. 5 shows an anchor 510 having a first member 525a and a second member 525b of a first stabilizer extending from a first ring 519 that fold inward when in a crimped state, as well as a first member 520a and a second member 520b of a second stabilizer, as well as a positioning arm 530 extending from a first ring 519 that fold inward when in a crimped state. FIG. 6 shows a hybrid combination in which the first and second members 625a, 625b of the first stabilizer on the first ring 619 coil inward while the first and second members 620a, 620b of the second stabilizer on the second ring 618 fold inward when the anchor 610 is in a crimped state.

FIG. 7 illustrates a ring 770 that may be used at the proximal or distal end of an anchor, having a plurality of stabilizer members 775a-d, which, in a crimped state are configured to bend inward as shown in FIG. 7. The stabilizer members of this embodiment may be configured to bend outward in a variety of shapes upon deployment of the anchor, as illustrated in FIG. 7A. Alternatively, the stabilizer extending from ring 870 may have a plurality of members each forming a loop—for example, first member 875a and second member 875b may each form a loop a continuous loop extending outward in a plurality of directions as illustrated by FIG. 8. FIG. 8A illustrates a top perspective of various embodiments of ring 870 having coiled stabilizer members in a deployed state. FIG. 17 illustrates yet another embodiment of the anchor 1710 in a fully deployed state.

As illustrated in the embodiments of FIGS. 9A-9C, the anchor bridge 915 of the anchor 910 may be formed of an elastic or flexible material capable of stretching or contracting to adjust to the dimensions of the vessel wall in which the anchor is deployed. Alternatively, the bridge may be preset at an angle such that the angle can be straightened upon deployment to accommodate the thickness of the tissue wall. In this embodiment, the preset angle or degree of bend in the bridge will define the thinnest tissue wall for secure implantation of the anchor, whereas the fully extended length of the bridge will define the thickest tissue wall. Upon crimping into the delivery system, the bridge may be straightened. Upon deployment, the anchor bridge will maintain a varying degree of contraction to accommodate the thickness of the body tissue wall at the implantation site.

FIG. 9A shows the anchor 910 in a semi-contracted state, FIG. 9B shows the anchor 910 in an extended state, and FIG. 9C shows the anchor in a contracted state. FIGS. 9D and 9E illustrate the anchor deployed in two different target tissues. Target tissue 920 of FIG. 9D is thinner than target tissue 930 of FIG. 9E. When deployed at target tissue 920, bridge 915 of the anchor may be contracted such that first and second stabilizers 940 and 950 are both in contact with the target tissue, illustrated in FIG. 9D. When deployed at target tissue 930, ridge 915 may be in a more extended state so that stabilizers 940 and 950 are also both in contact with the target tissue, illustrated in FIG. 9E. The elastic bridge 915 allows the anchor to be secured embedded in target tissues having different thicknesses.

The present invention also relates to a deployment system for the percutaneous delivery and secure implantation of an anchor in the target site wall. FIG. 10 illustrates an introducer cannula 1040 for percutaneous delivery of an anchor having an interior lumen 1041 and a tip 1042. The introducer cannula 1040 is adapted to house an anchor 1010, a sheath 1050, and a pushrod 1060. The cannula 1040 may comprise an outer diameter in the range between 1 to 50 G, an inner diameter in the range of 0.01 to 20 mm, a length of 1 to 200 cm, and comprises a suitable semi-flexible, biocompatible material for use within the body. Suitable materials include, for example, silicones, polyvinyl chloride (PVC) or other medical grade biocompatible polymers. In one particular embodiment, the introducer cannula 1040 has an outer diameter of 17 G, an inner diameter of 1.06 mm, a length of 20 cm and is made of a semi-flexible, biocompatible material. The anchor 1010 is designed such that, in a crimped state, the first stabilizer 1025 and the second stabilizer 1020 have a diameter of sufficient width to fit within the interior lumen 1041 of the introducer cannula 1040 without causing bulges.

The pushrod 1060 is contained within the interior lumen 1041 of the introducer cannula 1040 and directly abuts the anchor 1010. The pushrod 1060 may have an outer diameter in the range of less than 0.01 to no greater than 20 mm, a length in the range of 1 to 200 cm, and an inverted cone in the piston of the pushrod 1060, which is adapted to protect the area around the anchor 1010. The pushrod 1060 is adapted to move lengthwise inside the interior lumen 1041 of the introducer cannula 1040 from the proximal end of the interior cannula 1040 to the target implantation site to deploy the anchor 1010. The pushrod 1060 may be solid or hollow, and comprises a suitable semi-flexible biocompatible material, such as Nitinol, silicone, PVC, titanium or stainless steel. The materials of the introducer cannula 1040 and the pushrod 1060 may be same or different, provided that the pushrod 1060 is comprised of a material sufficiently rigid to resist collapse.

The anchor 1010 is oriented such that the first stabilizer 1025 and positioning arm 1030 are located at the distal end of the anchor 1010 near the tip 1042 of the cannula 1040, and the second stabilizer 1020 is located at the proximal end of the anchor 1010. In the embodiment illustrated by FIG. 10, wherein the anchor is delivered directly unto the target site using a cannula only (e.g. extra-luminally), the positioning arm 1130 is preferably oriented at the distal end of the anchor.

The sheath 1050 extends over the anchor 1010 and is adapted to be controllably retracted to release the anchor 1010 at the deployment site. The sheath 1050 may be retracted to deploy the anchor 1010 while the pushrod 1060 remains stationary to position the anchor 1010 at the target implantation site. The sheath 1050 may be manipulated by the operator directly or remotely, so that the anchor 1010 is released from the sheath 1050 at the discretion of the operator. For example, the sheath 1050 may extend from the anchor 1010 through the interior lumen 1041 of the introducer cannula 1040 to the proximal end, allowing the operator to manipulate the sheath 1050 directly through mechanical means. Alternatively, the stabilizers may also be releasable using shape-memory materials, for example, Nitinol or shape-memory polymers, which may be controllable by well-known means in the art, such as heat, light, chemical, pH, magnetic or electrical stimuli, described in, for example, U.S. Pat. No. 6,720,402 and U.S. Pat. No. 2009/0306767, both of which are incorporated by reference in their entirety. For example, the shape-memory material may be in a form of a spring, capable of contraction and expansion as an electric current is applied or removed. Electroactive polymers or magnetic shape memory alloys may also be employed in a similar fashion. Another alternative may be a remote control mechanism, such as for example, a string and loop-mechanism where the string is threaded through a loop or similar hoop structure on the sheath 1050, and the two ends of the string are located towards the proximal end of the introducer cannula 1040. To verify the secure embedding of the anchor, both ends of the string may be pulled to ensure the sheath 1050 is not dislodged. Releasing one end of the string unthreads the string from the loops, and the sheath 1050 may be retracted thereafter. The sheath 1050 may comprise any suitable size or shape to be arranged within the interior lumen 1041 of the introducer cannula 1040. The sheath 1050 may be formed of a braided polymer, such as polyimide, that may be braided together with a biocompatible material, such as a silicone, PVC, titanium or stainless steel. The sheath 1050 may be comparatively less rigid than the pushrod 1060.

The present invention also relates to a method of implanting an anchor into a target site wall. The method comprises protruding the cannula 1040 through the target site wall 1080, as illustrated by FIG. 10. FIG. 10A illustrates the anchor 1010 in an early stage of deployment wherein an application of force to the pushrod 1060 extends the anchor 1010 into the target site 1090, retracting sheath 1050 from the anchor 1010 and deploying the first stabilizer 1025 and positioning arm 1030. The first stabilizer 1025 and the positioning arm 1030 revert to a deployed state within the target site 1090, while the bridge 1015 traverses the target site wall 1080. The deployed state of the first stabilizer 1025 may be any shape that expands the area of the coil in a direction perpendicular to the axis of the bridge, i.e., generating a configuration that prevents the anchor from dislodging from its position in the target site wall. The semi-deployed anchor 1010 may then be tugged gently in the counter direction (i.e. pulled snug against the target site wall) to ensure that the anchor 1010 is properly embedded in the vessel prior to full deployment. FIG. 10B illustrates the anchor 1010 in a state of full deployment following full retraction of the sheath 1050 and cannula 1040, thereby deploying the second stabilizer 1020 from a crimped state and permitting the second stabilizer 1020 to unspool partially. In the deployment stage, each of the first and second stabilizers form a flattened coil configuration. Thus, the first stabilizer 1025 unspools along the outer wall 1081 of the target site, the anchor bridge 1015 traverses the target site wall 1080, the second stabilizer 1020 unspools along the inner wall 1082 of the target site, and the positioning arm 1030 reverts to a preselected position to orient the anchor in a specific orientation relative to the target site, according to design specifications. The introducer cannula 1040, sheath 1050 and pushrod 1060 are fully retracted from the anchor 1010.

Preferably, the sheath has a feedback mechanism that assures the anchor is securely implanted prior to the retraction of the pushrod. In one embodiment, feedback is provided to the operator by resistance of the unsheathed first ring against the inner wall of the vessel at the target implantation site. Mechanical resistance to retracting the anchor signals to the operator that the anchor is successfully deployed within the inner portion of the vessel and that the second ring may be unsheathed to fully deploy the anchor.

The force feedback mechanism may be adapted to the user-controlled sheath described above. In another embodiment, a force meter may be used with the sheath to ensure that the anchor is securely deployed at the target site. The force meter may be used to measure the degree of force of the first stabilizer in a deployment position against the interior vessel wall upon partial deployment of the anchor, thus signal to the operator that the second stabilizer may be unsheathed for full deployment. The force meter also may be used to measure the degree of pushing force used to pierce the vessel wall, as well as the amount of pulling strain demonstrated by the anchor to ensure that the anchor will remain engaged within the body lumen and not prematurely dislodge. One example of a force meter that may be incorporated within the system of this invention is described in U.S. Pub. No. 2010/0024574, the contents of which are incorporated herein by reference.

In one embodiment, illustrated by FIG. 11, the pushrod 1160 may be hollow and house the anchor 1110 within the pushrod 1160 up to the first ring 1119. The pushrod 1160 comprises an aperture 1161 through which the second stabilizer 1120 of the anchor 1110 extends. As further illustrated by FIG. 12A, the sheath 1250 covers the outside of the pushrod 1260, thus forming a release mechanism 1280 that compresses the portion of the second stabilizer 1220 protruding through the aperture 1261 between the pushrod 1260 and the sheath 1250. The release mechanism is advantageous in preventing premature release of the stabilizer prior to deployment at the target site. FIG. 12B illustrates the result of retracting or releasing the sheath 1250 from the pushrod 1260, whereby the second stabilizer 1220 is released from the compression.

FIGS. 13A-B illustrate another embodiment of the release mechanism 1380, whereby the aperture of the pushrod 1360 comprises an “L”-shape slit formed by an aperture arm 1361a and an aperture neck 1361b. In the deployment position, the second stabilizer 1320 extends through the aperture arm 1361a as illustrated by FIG. 13A. Upon rotation of the pushrod 1360, the aperture neck 1361b aligns with the second stabilizer 1320, as illustrated by FIG. 13B, thus permitting the deployment of the second stabilizer 1320 upon retraction of the sheath 1350. Rotation of the pushrod 1360 around the second stabilizer 1320 is possible due to the frictional force of the first stabilizer once deployed against the opposite-facing wall of the target site.

The deployment system described above may be used to implant the anchor in any accessible tissue wall of the body, such as in the cardiovascular system, the hepatic-portal venous system, or in the gastrointestinal tract. In one embodiment, the invention may be useful in the hepatic-portal venous systems during portal venous catheterization procedures to implant the anchor in the portal vein. In another embodiment, arteries of the cardio-vascular system can be monitored through implantation of a small implantable element in an artery or in certain veins.

The small implantable element may monitor any bodily characteristic within a body cavity or lumen. Examples of such elements measure physical or chemical characteristics of the body, such as, for example, sensors, monitors, attenuators, or regulators of luminal or extraluminal function. Alternatively, the small implantable element may treat a medical condition, for example, by releasing a therapeutic agent.

In any of the embodiments above, the anchor may comprise a radiopaque marker attached to a component of the anchor, e.g., the positioning arm. Alternatively, the anchor, partly or in whole, may be composed of a radiopaque material. Radiopaque material may include gold, boron, tantalum, platinum iridium or similar materials. The use of radio-opaque materials allows visualization by x-ray or patterned with ultrasonic grating, or both.

The invention further relates to a method of manufacturing an anchoring system, comprising the steps of producing a wire from a suitable material, such as, for example, Nitinol; and applying a heat treatment to the wire to conform the wire into the shape of a flattened coil to thermomechanically preset the stabilizer's deployed configuration. Other methods of manufacturing according to the invention includes, for example, laser cutting, chemical etching, electrochemical machining, electrical discharge machining, or other traditional machine methods. The invention may be manufactured from a flat metal sheet or a stock tube, such as, for example, Nitinol or stainless steel, into a preselected pattern. The pattern thus can be coiled through application of heat treatment, welding or mechanical force.

FIG. 14 illustrates a pattern of the anchor 110 for example as shown in FIG. 1A, having a bridge 115 with a second end 116 and a first end 117. A second band 111 occurs at the second end 116 of the bridge 115 with a second stabilizer 120 extending therefrom in a direction parallel to the bridge 115. The second band 111 may be formed into a ring and welded together, and upon heat treatment or other means, the second stabilizer 120 may be configured into a pre-deployment state. A first band 112 occurs at the first end 117 of the bridge 115 with a first stabilizer 125, as well as positioning arm 130 extending therefrom in a direction parallel to the anchor bridge 115.

The positioning arm 130 may be located in various positions around the first band 112, for example as illustrated, out of alignment with the bridge 115. The first band 112 may be formed into a ring and welded together, and upon heat treatment or other means, the first stabilizer 125 may be configured into a pre-deployment state. FIG. 15 illustrates a laser-cut pattern of the anchor for example, as shown in FIG. 2A, having a first member 225a and a second member 225b of the first stabilizer positioned 180 degrees apart around the ring, as well as a first member 220a and a second member 220b of the second stabilizer similarly positioned. Alternatively, the members may be positioned variously around the band. In yet another alternative, the ends of the members may be connected to form a continuous loop. FIG. 16 illustrates yet another laser-cut pattern further including a third member 220c of the second stabilizer. Similarly other embodiments having varying numbers of members variously positioned along the bands will be readily apparent to the skilled person and are within the scope of the invention.

FIG. 18A illustrates a mandrel 1800 having a first disc 1810, a second disc 1820 and an axel 1830 therebetween. As illustrated by FIG. 18B, the first disc 1810 has a first surface 1815 that is convex towards the second disc 1820, and the second disc 1820 likewise has a second surface 1825 that is convex towards the first disc 1810. Although FIG. 18A shows convex surfaces on the first and second discs, the invention contemplates other surface shapes, including flat or concave surfaces. On each first and second surfaces 1815, 1825, grooves 1816, 1826, respectively, spiral away from axel 1830 toward the edges of the discs 1810, 1820.

Mandrel coverings are configured to encase mandrel 1800, which may be of any shape or size. As illustrated in FIG. 18C, one mandrel covering 1850 is designed to encase roughly half of the surface area of axel 1830 and first or second surfaces 1825 or 1826. Mandrel covering comprises axel covering portion 1860. FIG. 18D shows two mandrel coverings that encase substantially the entirety of axel 1830 and first and second surfaces 1825 and 1826. While FIGS. 18C and 18D illustrate the two mandrel coverings 1850 that are symmetrical, this invention contemplates that the number, shape and size of the mandrel coverings are not limited to the illustrated embodiments. Mandrel coverings 1850 may be secured around the mandrel by any securement means in the art, including latches or clasps. Further, a wire may be spindled around axel covering portion 1860 to keep mandrel covering 1850 secure on the mandrel.

In the manufacturing process, a material, e.g., a wire, forming the anchor is placed in the groove of the mandrel. Once placed, the mandrel coverings are secured onto the mandrel, thereby restricting the movement of the material. The material-loaded mandrel, along with the mandrel coverings are then heat treated as known in the art, such that the material retains the shape of the groove of the mandrel. Materials placed in the grooves of the first and second surfaces form the first and second stabilizers, while materials in contact with the axel form the bridge and first and second rings. Preferably, the material forming the ring does not join in a circular shape when placed onto the mandrel, allowing for the anchor to be removed after the heat treatment.

In manufacturing the embodiment having an extendable bridge, such as, for example, the embodiments of the anchor illustrated in FIG. 9A-9E, the mandrel may comprise a first disc, a second disc and a axel therebetween. The axel of this embodiment may be configured with one or a plurality of bends such that when the material is placed onto the mandrel and heated, the resulting bridge has bend(s) in the relaxed state in accordance with the shape of the axel. Similarly, the shape and size of the mandrel coverings may be modified to complement the profile of the axel of this mandrel.

Following the formation of the anchor as described above, the anchor is removed from the mandrel, allowing for further manufacturing processes, including welding, soldering, brazing or attachment of additional components, e.g., joining of the first and second rings, attaching a positioning arm, or attaching a small implantable element.

It will be appreciated by persons having ordinary skill in the art that many variations, additions, modifications, and other applications may be made to what has been particularly shown and described herein by way of embodiments, without departing from the spirit or scope of the invention. Although the invention has been particularly shown and described herein by way of embodiments, it will be appreciated by persons having ordinary skill in the art that also various kinds of combinations of these embodiments or combinations of specific features of these embodiments may be made without departing from the spirit or scope of the invention. Therefore, it is intended that the scope of the invention, as defined by the claims below, includes all foreseeable variations, additions, modifications, or applications.

1. An implantable anchor comprising a proximal stabilizer, a distal stabilizer, a bridge therebetween and a positioning arm, wherein said first and second stabilizers have a crimped configuration and are capable of a deployed configuration.

2. The anchor of claim 1, wherein the positioning arm has a crimped state and a deployed state.

3. The anchor of claim 1, wherein the a small implantable element is attached to said positioning arm.

4. The anchor of claim 3, wherein the small implantable element is a sensor.

5. The anchor of claim 4, wherein said sensor is adapted to monitor blood pressure.

6. The anchor of claim 4, wherein said sensor is adapted to monitor chemical characteristics.

7. The anchor of claim 1, further comprising a first ring and a second ring.

8. The anchor of claim 7, wherein the first stabilizer and the positioning arm extend from the first ring, and the second stabilizer extends from the second ring.

9. The anchor of claim 7, wherein the first ring is larger than the second ring.

10. A deployment system for percutaneously delivering and implanting an anchor having a first stabilizer, a second stabilizer and a bridge therebetween having a crimped configuration and capable of a deployed configuration, comprising an introducer cannula, a pushrod, and a sheath.

11. The deployment system of claim 10 wherein the anchor comprises a bridge having a first ring and a second ring, wherein said first stabilizer and a positioning arm extend from the first ring and said second stabilizer extends from the second ring.

12. The deployment system of claim 10 wherein the sheath maintains the first stabilizer, second stabilizer and positioning arm in a crimped state prior to deployment.

13. The deployment system of claim 10, further comprising a needle.

14. A method for using a deployment system comprising a cannula, sheath and anchor, said anchor having a positioning arm, a first stabilizer extending from a first ring, a second stabilizer extending from a second ring oriented, and said sheath maintains the first stabilizer and the second stabilizer in a crimped state, said method comprising the steps of:
advancing said cannula to a target site;
positioning the anchor at the target site;
administering a controlled amount of force to release the first stabilizer from a crimped state;
administering a controlled amount of force to release the second stabilizer from a crimped state and
retracting said cannula.

15. The method of claim 14, said target site being a location in the hepatic portal vein.

16. The method of claim 14, wherein said sheath comprises a mechanical means for controllably releasing the first stabilizer, the second stabilizer and the positioning arm from said crimped state.

17. The method of claim 14, wherein the deployment system further comprises a pushrod, comprising the steps of:
inserting said pushrod through said cannula to advance the anchor to the target site, and
retracting said pushrod following deployment of the anchor.

18. The method of claim 14, wherein said positioning arm extends from the first ring.

19. The method of claim 14, wherein the positioning arm extends from the second ring.

20. The method of claim 14, wherein the deployment system further comprises a needle and, before advancing said cannula to a target site, the method further comprises:
piercing an outer body tissue with said needle; and
advancing said cannula over said needle.

21. A method of manufacturing an anchor, comprising the steps of:
placing a material on a mandrel;
covering the mandrel with a mandrel covering; and
applying a heat treatment to said material to form said anchor.

22. The method of claim 21 further comprising a mandrel having a first disc, a second disc and an axel, wherein the first disc and second disc each have a spiral groove.

23. A mandrel for manufacturing an anchor having expandable first and second stabilizers comprising a first disc, a second disc and an axel therebetween, said first disc having a convex surface toward the second disc and said second disc having a convex surface toward the first disc, wherein each disc has a groove extending from the axel.

24. The mandrel according to claim 23, further comprising a mandrel covering having a plurality of parts that together encase the mandrel.
